SEQUENCE LISTING (1 sequence)

SEQ ID NO: 1 - protein, 920 aa, Homo sapiens
MSDPHSSPLLPEPLSSRYKLYEAEFTSPSWPSTSPDTHPALPLLEMPEEKDLRSSNEDSHIVKIEKLNERSKRKDDGVAHRDSAGQRCICLSKAVGYLTGDMKEYRIWLKDKHLALQFIDWVLRGTAQVMFINNPLSGLIIFIGLLIQNPWWTITGGLGTVVSTLTALALGQDRSAIASGLHGYNGMLVGLLMAVFSEKLDYYWWLLFPVTFTAMSCPVLSSALNSIFSKWDLPVFTLPFNIAVTLYLAATGHYNLFFPTTLVEPVSSVPNITWTEMEMPLLLQAIPVGVGQVYGCDNPWTGGVFLVALFISSPLICLHAAIGSIVGLLAALSVATPFETIYTGLWSYNCVLSCIAIGGMFYALTWQTHLLALICALFCAYMEAAISNIMSVVGVPPGTWAFCLATIIFLLLTTNNPAIFRLPLSKVTYPEANRIYYLTVKSGEEEKAPSGGGGEHPPTAGPKVEEGSEAVLSKHRSVFHIEWSSIRRRSKVFGKGEHQERQNKDPFPYRYRKPTVELLDLDTMEESSEIKVETNISKTSWIRSSMAASGKRVSKALSYITGEMKECGEGLKDKSPVFQFFDWVLRGTSQVMFVNNPLSGILIILGLFIQNPWWAISGCLGTIMSTLTALILSQDKSAIAAGFHGYNGVLVGLLMAVFSDKGDYYWWLLLPVIIMSMSCPILSSALGTIFSKWDLPVFTLPFNITVTLYLAATGHYNLFFPTTLLQPASAMPNITWSEVQVPLLLRAIPVGIGQVYGCDNPWTGGIFLIALFISSPLICLHAAIGSTMGMLAALTIATPFDSIYFGLCGFNSTLACIAIGGMFYVITWQTHLLAIACALFAAYLGAALANMLSVFGLPPCTWPFCLSALTFLLLTTNNPAIYKLPLSKVTYPEANRIYYLSQERNRRASIITKYQAYDVS

(19) United States
(12) Patent Application Publication
(10) Pub. No.: US 2021/0106547 A1
(43) Pub. Date: Apr. 15, 2021

(54) Managing Cardiovascular Conditions Associated with Kidney Disease

(71) Applicant: Emory University, Atlanta, GA (US)

(72) Inventors: Jeff M. Sands, Atlanta, GA (US); Janet D. Klein, Atlanta, GA (US); Xiaonan H. Wang, Atlanta, GA (US)

(21) Appl. No.: 17/067,477

(22) Filed: Oct. 9, 2020

Related U.S. Application Data
(60) Provisional application No. 62/913,479, filed on Oct. 10, 2019.

(51) Int. Cl. A61K 31/17; A61K 31/585; A61K 31/341

(52) U.S. Cl. CPC A61K 31/17; A61K 31/341; A61K 31/585

(57) ABSTRACT

This disclosure relates to managing cardiovascular diseases and conditions associated with kidney disease. In certain embodiments, this disclosure relates to methods of treating or preventing a cardiovascular disease or condition comprising administering an effective amount of a urea transporter inhibitor to a subject in need thereof. In certain embodiments, the subject is diagnosed with kidney disease. In certain embodiments, the subject is diagnosed with uremic cardiomyopathy. In certain embodiments, the urea transporter inhibitor is N,N′-dimethylthiourea (DMTU), prodrug, derivative, or salt thereof.

### CROSS-REFERENCE TO RELATED APPLICATIONS

This application claims the benefit of U.S. Provisional Application No. 62/913,479 filed Oct. 10, 2019. The entirety of this application is hereby incorporated by reference for all purposes.

### STATEMENT REGARDING FEDERALLY SPONSORED RESEARCH OR DEVELOPMENT

This invention was made with government support under DK041707 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 19207US_ST25.txt. The text file is 9 KB, was created on Oct. 8, 2020, and is being submitted electronically via EFS-Web.

### BACKGROUND

The prevalence of cardiovascular disease in chronic kidney disease (CKD) patients reaches 65% in the population aged 66 and older, while the prevalence of cardiovascular disease is 32% those without CKD. Many CKD patients die within three years from the time of being diagnosed with uremic cardiomyopathy. Uremic cardiomyopathy mainly consists of left ventricular hypertrophy and interstitial fibrosis. Cardiac fibrosis is a constant finding in heart biopsies and necropsy studies in CKD patients, which is related to retention of water, sodium, and urea within the body. Thus, there is a need to identify methods for managing cardiovascular conditions in patients with kidney disease.

Duchesne et al. report UT-A urea transporter protein in heart increased in abundance during uremia, hypertension, and heart failure. Circ Res. 2001, 89:139-145. Verkman et al. report small-molecule inhibitors of urea transporters. Subcell Biochem. 2014, 73: 165-177. Cil et al. report a diuretic effect of dimethylthiourea in female rats. Hum Exp Toxicol. 2012, 31(10):1050-5. Esteva-Font et al. report thiourea analogs as inhibitors of UT-A and UT-B urea transporters. Biochim Biophys Acta. 2015, 1848(5):1075-80. See also US Patent Application No. 2017/0239235.

References cited herein are not an admission of prior art.

### SUMMARY

This disclosure relates to managing cardiovascular conditions associated with kidney disease. In certain embodiments, this disclosure relates to methods of treating or preventing a cardiovascular condition comprising administering an effective amount of a urea transporter inhibitor to a subject in need thereof. In certain embodiments, the subject is diagnosed with kidney disease. In certain embodiments, the subject is diagnosed with uremic cardiomyopathy. In certain embodiments, the urea transporter inhibitor is N,N′-dimethylthiourea (DMTU), prodrug, derivative, or salt thereof.

In certain embodiments, the urea transporter inhibitor is small molecule, protein, antibody or fragment thereof that specifically binds urea transport protein A and/or urea transport protein B. In certain embodiments, the urea transporter inhibitor is administered in combination with a statin, antihypertensive agent, diuretic, or combination thereof. In certain embodiments, the diuretic is spironolactone or furosemide. In certain embodiments, the antihypertensive agent is an angiotensin receptor II blocker, angiotensin-converting enzyme inhibitor, calcium channel blocker, beta blocker, or combinations thereof. In certain embodiments, the subject is over 50, 60, or 65 years of age.

In certain embodiments, this disclosure relates to pharmaceutical composition comprising a urea transporter inhibitor and a pharmaceutically acceptable excipient. In certain embodiments, the urea transporter inhibitor is N,N′-dimethylthiourea (DMTU), prodrug, derivative, or salt thereof. In certain embodiments, the urea transporter inhibitor is small molecule, protein, antibody or fragment thereof that specifically binds urea transport protein A and/or urea transport protein B. In certain embodiments, the pharmaceutical composition further comprises a statin, antihypertensive agent, diuretic, or combination thereof In certain embodiments, the antihypertensive drug is an angiotensin receptor II blocker, angiotensin-converting enzyme inhibitor, calcium channel blocker, beta blocker, or combinations thereof. In certain embodiments, the diuretic is spironolactone or furosemide.

### BRIEF DESCRIPTION OF THE SEVERAL VIEWS OF THE DRAWINGS

FIGS. 1A-1E show data indicating cardiac hypertrophy and fibrosis appeared in the 5/6th nephrectomy mouse model.

FIG. 1A shows data on sham and 5/6Nx mice were sacrificed and samples were harvested at 8 weeks after the second surgery. Systolic and diastolic blood pressure (BP) were measured by tail cuff method in sham and 5/6Nx mice.

FIG. 1B shows pictures of mouse heart at sacrifice.

FIG. 1C shows heart weight (mg) normalized by body weight (BW) (g).

FIG. 1D shows Masson's Trichrome staining in mouse heart tissues. Fibrosis is indicated by dark color. Pictures were observed by ×200 magnification. Scale bar is 100 μm.

FIG. 1E shows quantitative mRNA expression in mouse hearts performed by real-time PCR. Individual gene expression was calculated by ΔΔcq and standardized by housekeeping gene 18s.

FIGS. 2A-2E show data indicating cardiac fibrosis in uremic heart was associated with upregulation of UT-A and vimentin expression.

FIG. 2A shows western blot of lysates from C57BL/6 wild type mouse hearts for UT-A protein. Heart lysates were probed with (left) anti-UT-A antibody and (right) anti-UT-A antibody that was pre-adsorbed with immunizing peptide of UT-A. 20 ug protein (left lane) and 40 ug protein (right lane) were loaded.

FIG. 2B shows immunofluorescence images for UT-A/GFP in H9c2 cells. Left panel are control H9c2 cells without transfection and right panel are H9c2 cells transduced with adenovirus (ad)-UT-A/GFP. Pictures of these cells were taken 48 hours after virus transduction.

FIG. 2C shows western blot of lysates from H9c2 cells 48 hours after transduction.

FIG. 2D shows western blot of lysates from sham and 5/6Nx mouse hearts probed for UT-A and vimentin proteins.

FIG. 2E shows relative density.

FIGS. 3A-3E shows data indicating inhibition of UT-A by dimethylthiourea (DMTU) ameliorated CKD-induced hypertension and cardiac hypertrophy.

FIG. 3A shows data where blood pressure measurement of mice was performed by tail cuff method, and heart rate was measured together with blood pressure. Systolic blood pressure at 8 weeks after the second surgery.

FIG. 3B shows diastolic blood pressure.

FIG. 3C shows pictures of mouse hearts at sacrifice.

FIG. 3D shows heart weight (mg) normalized by body weight (g) at sacrifice.

FIG. 3E shows heart dry weight (mg) normalized by brain dry weight (mg). White bar is vehicle and black bar is DMTU treatment.

FIGS. 4A-4E shows data indicating inhibition of UT-A attenuated CKD-induced cardiac fibrosis.

FIG. 4A shows Masson-Trichrome staining of mouse heart tissues. Pictures were observed by ×200 magnification. Scale bar is 100 μm. Dark color indicated fibrosis in Masson's staining.

FIG. 4B shows immunohistochemistry staining for collagen type 1 at ×400 magnification. Scale bar is 50 μm.

FIG. 4B shows immunohistochemistry staining for α-SMA. Scale bar is 50 μm.

FIG. 4D shows the percentage of fibrosis area per entire area was calculated by computed software.

FIG. 4E show quantitative mRNA expression in mouse hearts performed by real-time PCR. Individual gene expression was calculated by ΔΔcq and standardized by housekeeping gene 18s.

FIG. 5 shows data indicating inhibition of UT-A suppressed CKD-induced upregulation of angiotensin converting enzyme: Quantitative mRNA expression in mouse hearts performed by real-time PCR. Angiotensin converting enzyme (ACE) gene expression was calculated by ΔΔcq and standardized by housekeeping gene 18s.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

The human UT-A gene (SLC14A2) is located on chromosome 18 and is approximately 67.5 kb in length. The human UT-A gene gives rise to UT-A1, UT-A2, and UT-A3 and to a shorter isoform, UT-A6. UT-A2 and UT-A1 have the same C-terminal amino acid (and 3′ cDNA) sequence but differ at the N-terminal (5′ cDNA) end. Hence, UT-A2 is essentially the C-terminal half of UT-A1 (amino acids 1-532 missing). Human UT-A1 protein is (SEQ ID NO: 1)

MSDPHSSPLLPEPLSSRYKLYEAEFTSPSWPSTSPDTHPALPLLEMPEEKD
LRSSNEDSHIVKIEKLNERSKRKDDGVAHRDSAGQRCICLSKAVGYLTGDM
KEYRIWLKDKHLALQFIDWVLRGTAQVMFINNPLSGLIIFIGLLIQNPWWT
ITGGLGTVVSTLTALALGQDRSAIASGLHGYNGMLVGLLMAVFSEKLDYYW
WLLFPVTFTAMSCPVLSSALNSIFSKWDLPVFTLPFNIAVTLYLAATGHYN
LFFPTTLVEPVSSVPNITWTEMEMPLLLQAIPVGVGQVYGCDNPWTGGVFL
VALFISSPLICLHAAIGSIVGLLAALSVATPFETIYTGLWSYNCVLSCIAI
GGMFYALTWQTHLLALICALFCAYMEAAISNIMSVVGVPPGTWAFCLATII
FLLLTTNNPAIFRLPLSKVTYPEANRIYYLTVKSGEEEKAPSGGGGEHPPT
AGPKVEEGSEAVLSKHRSVFHIEWSSIRRRSKVFGKGEHQERQNKDPFPYR
YRKPTVELLDLDTMEESSEIKVETNISKTSWIRSSMAASGKRVSKALSYIT
GEMKECGEGLKDKSPVFQFFDWVLRGTSQVMFVNNPLSGILIILGLFIQNP
WWAISGCLGTIMSTLTALILSQDKSAIAAGFHGYNGVLVGLLMAVFSDKGD
YYWWLLLPVIIMSMSCPILSSALGTIFSKWDLPVFTLPFNITVTLYLAATG
HYNLFFPTTLLQPASAMPNITWSEVQVPLLLRAIPVGIGQVYGCDNPWTGG
IFLIALFISSPLICLHAAIGSTMGMLAALTIATPFDSIYFGLCGFNSTLAC
IAIGGMFYVITWQTHLLAIACALFAAYLGAALANMLSVFGLPPCTWPFCLS
ALTFLLLTTNNPAIYKLPLSKVTYPEANRIYYLSQERNRRASIITKYQAYD
VS.

Polyclonal antibodies have been raised against three portions of UT-A1: the N-terminus, the intracellular loop region, and the C-terminus. These antibodies often detect more than one UT-A protein since the various UT-A proteins result from alternative splicing of the UT-A gene. However, they can be distinguished by the different size bands that are detected on Western blot. The N-terminus antibody detects UT-A1 (97 and 117 kDa), UT-A3 (44 and 67 kDa), and UT-A4 (43 kDa); the loop region antibody detects only UT-A1; and the C-terminus antibody detects UT-A1, UT-A2 (55 kDa), and UT-A4. In the inner medullary tip region, all anti-UT-A1 antibodies detect protein bands at 97 and 117 kDa by Western blot. The 97- and 117-kDa proteins are glycosylated versions of a non-glycosylated 88-kDa UT-A1 protein.

Inhibition of Urea Transporter Ameliorates Uremic Cardiomyopathy in Mice with Chronic Kidney Disease

The prevalence of cardiovascular disease in chronic kidney disease (CKD) patients reaches 65%, compared with 32% without CKD, in the population aged 66 and older. Many CKD patients die within three years from the time of being diagnosed with uremic cardiomyopathy. Uremic cardiomyopathy mainly consists of left ventricular hypertrophy and interstitial fibrosis that is associated with hospitalization and the risk of death. Cardiac fibrosis is a constant finding in heart biopsies and necropsy studies in CKD patients, which is related to retention of water, sodium, and urea within the body. An increase in cardiac fibrosis is also associated with hypertension and activation of the renin-angiotensin system (RAS) in 5/6 nephrectomy (5/6Nx) mice. Activation of RAS and volume overload contribute to cardiac hypertrophy and blocking the type I angiotensin II receptor suppresses left ventricular hypertrophy.

Urea plays a major role in the urine concentrating mechanism. UT-A1 and UT-A3 are located in the kidney inner medullary collecting duct (IMCD). In UT-A1/A3−/− mice, urine volume is increased and plasma urea nitrogen (BUN) is low compared with wild type mice, even if they were fed a high protein (40%) diet. These mice also have a decreased ability to concentrate their urine. Similar effects are seen with inhibitors of UT-A. So, an inhibition of UT may be useful to prevent volume overload and a high level of BUN and could show positive cardiac effects.

UT-A2 protein levels are increased in hypertensive or uremic heart. UT-A2 knock-out mice have an impaired ability to concentrate urine. Surprisingly, deletion of UT-A2 in UT-B knock-out mice appears to raise a urine osmolality. So, UT-A2 potentially accumulates urea in the outer medulla. UT-A2 protein in liver is associated with the production of urea from arginine via the polyamine pathway, but a role of UT-A2 in uremic heart is not understood.

UT inhibitors are potentially novel diuretics. Dimethylthiourea (DMTU), a urea analog, inhibits UT-A1 and UT-B. Rats treated with DMTU had a greater diuresis than those treated with furosemide, and DMTU did not show side effects like hyponatremia or hypokalemia. So, ‘urearetics’ that target UT-A1 may be effective diuretics and have less risk for adverse effects on electrolytes.

There is a need to find therapeutic strategies to attenuate uremic cardiomyopathy that may provide a particular cardio-protective benefit in CKD. CKD mouse model 5/6Nx was used to determine whether inhibition of UT-A may reduce hypertension, cardiac hypertrophy and fibrosis in these mice. Experiments reported herein indicate improved methods for the treatment of cardiomyopathy.

Cardiovascular disease is a common event that leads to a lower quality of life and a high risk of mortality in CKD patients. Uremic cardiomyopathy generally consists of cardiac hypertrophy and fibrosis induced by hypertension and volume overload. Although recent studies show that several hypertrophic factors, such as cytokines, growth factors, or hormones, may play a role in cardiomyopathy and cardiac fibrosis, the RAS and volume overload also contribute to the development of uremic cardiomyopathy. Resolving volume overload suppresses cardiac hypertrophy in end-stage kidney disease patients. To date, the beneficial therapies for cardiomyopathy in CKD patients are antihypertension drugs and diuretics such as ACE inhibitors, angiotensin receptor blockers, spironolactone, furosemide, or Ca2+ channel blockers. However, there are some reports of these therapies having insufficient benefits or harmful side effects. Therefore, there is a fundamental need to find improved targets of therapy for uremic cardiomyopathy. Experiments reported herein indicate that inhibition of UT-A suppresses the increase BP and volume retention and might prevent cardiac hypertrophy and fibrosis in subjects with CKD.

The pathogenesis of uremic cardiomyopathy in CKD patients seems to reflect a complex mechanism that, by current understanding, involves a combination of volume overload, hypertension, RAS, anemia, hyperphosphatemia, Klotho deficiency, and fibroblast growth factor. Volume overload is well known as a risk factor for mortality in CKD and end stage renal disease patient. Volume overload and hypertension lead to the development of left ventricular hypertrophy to maintain wall stress. Amelioration of hypervolemia and hypertension is a key step in preventing uremic cardiomyopathy.

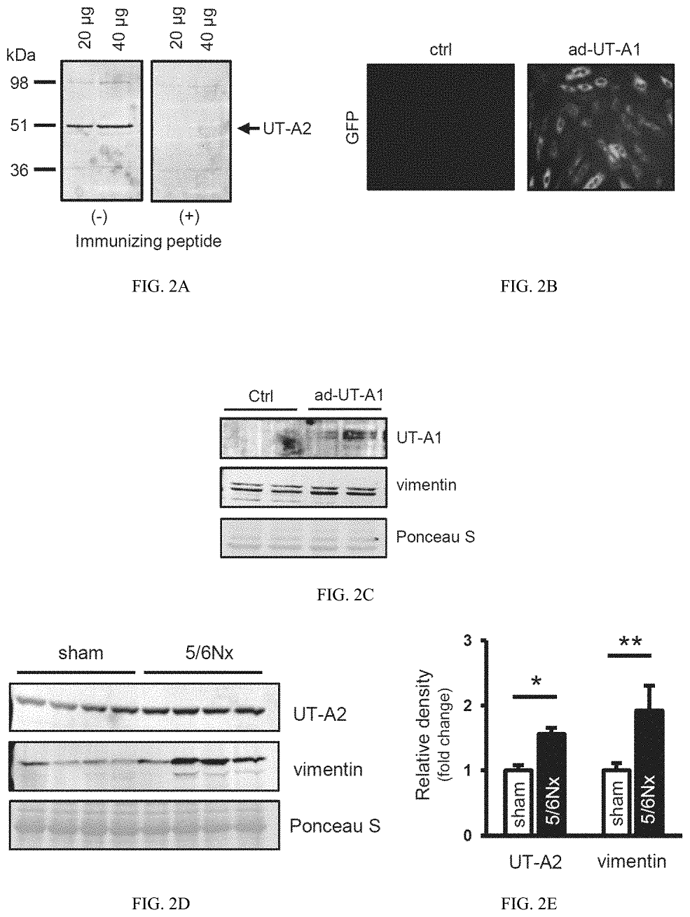

UT-A and aquaporin (AQP) 2 are the major transporters that support increased absorption of urea and water in the kidney collecting duct. Knockout of UT-A1 and UT-A3 is sufficient to cause polyuria, even in the presence of apparently normal AQP2. The UT-A1/A3−/− mice have urine volumes that are increased about 3-fold and urine osmolality that is decreased to about 35% compared with wild type mice on a normal 20% protein diet. The current study revealed an increase in a 51-kDa UT-A protein band in uremic heart tissue. UT-A1, which is the largest form of UT-A, shows 97- and 117-kDa glycoprotein bands upon electrophoretic separation. The UT-A2 protein consists of 397 amino acids that are identical to the sequence in the c-terminus of UT-A1. It is believed that UT-A2 is located in the heart, since a 51-kDa band was detected by UT-A antibodies in heart. Pretreating the antibodies with immunizing UT-A c-terminal peptide completely ablated this protein band (FIG. 2A). Urea is produced in the process of converting arginine to ornithine (urea cycle) in liver via the polyamine pathway, which is present in heart and associated with cardiac hypertrophy. Increased UT-A2 expression is associated with vimentin expression in H9c2 rat cardio myoblast cells (FIG. 2C). Since vimentin is a profibrotic protein linked with epithelial mesenchymal transition, it is believed that the increased levels of UT-A2 in the CKD heart may indicate a relationship with CKD induced cardiac fibrosis.

UT-A is important for the adjustment of extracellular fluid and intravascular volume. The abundance of UT-A decreased in volume expanded rats treated with aldosterone and high salt. A decrease in UT-A abundance is observed in the 5/6Nx rat model. Thus, a decrease of UT-A would potentiate extracellular volume expansion. In experiments reported herein, the urine volume of CKD groups increased compared with sham groups. Although the abundance of UT-A decreased in 5/6Nx model, our 5/6Nx+vehicle showed that the volume in-out balance slightly increased relative to that of sham+vehicle, and the balance was less in 5/6Nx+DMTU (Table 2). Thus, it appears that DMTU helps to prevent excessive volume retention.

Ventricular hypertrophy is a physio-pathological response to chronic hypertension and contributes to chronic heart failure and mortality. The local activation of the RAS in heart is associated with cardiac hypertrophy and fibrosis. Reports show that the expression of ACE increased in hearts with cardiac hypertrophy and fibrosis. Angiotensin II stimulates release of endothelin-1 from endothelial cells and endothelin-1 is strongly associated with cardiac hypertrophy and fibrosis. ACE-inhibitors prevent mRNA expression of ACE in heart and suppress cardiac hypertrophy and fibrosis. In experiments reported herein, DMTU suppressed an increase in ACE mRNA expression in CKD mice.

In summary, inhibition of UT-A attenuated both cardiac hypertrophy and cardiac fibrosis resulting from CKD. UT-A inhibitors such as DMTU have the ability to reduce volume retention and suppress hypertension and RAS activity even when kidney function declines. This suggests that UT-A inhibitors may be attractive therapeutic option for preventing uremic cardiomyopathy.

### Methods of Use

In certain embodiments, this disclosure relates to methods of treating or preventing a cardiovascular disease or condition comprising administering an effective amount of a urea transporter inhibitor to a subject in need thereof. In certain embodiments, the subject is diagnosed with kidney disease. In certain embodiments, the subject is diagnosed with uremic cardiomyopathy. In certain embodiments, the urea transporter inhibitor is N,N′-dimethylthiourea (DMTU), prodrug, derivative, or salt thereof.

In certain embodiments, the urea transporter inhibitor is small molecule, protein, antibody or fragment thereof that specifically binds urea transport protein A and/or urea transport protein B. In certain embodiments, the urea transporter inhibitor is administered in combination with statin, antihypertensive agent, diuretic, or combination thereof. In certain embodiments, the diuretic is spironolactone or furosemide. In certain embodiments, the antihypertensive agent is an angiotensin receptor II blocker, angiotensin-converting enzyme inhibitor, calcium channel blocker, beta blocker, or combinations thereof. In certain embodiments, the subject is over 50, 60, or 65 years of age.

The term “subject” refers to any animal, preferably a human patient, livestock, rodent, monkey or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, acid and the like.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

The term, “statin,” refers to drugs that are HMG-CoA reductase inhibitors which results in lowing lipid concentrations. Examples include atorvastatin, fluvastatin, lovastatin, pitavastatin, pravastatin, rosuvastatin, and simvastatin.

The terms, “antihypertensive” refers to drugs that reduce blood pressure such as, diuretics, calcium channel blockers, angiotensin-converting-enzyme inhibitors (ACE inhibitors), angiotensin II receptor antagonists (ARBs), and beta-adrenergic blocking agents (beta blockers). Examples of diuretics include bumetanide, ethacrynic acid, furosemide, torsemide, amiloride, spironolactone, eplerenone, triamterene, potassium canrenoate, bendroflumethiazide, hydrochlorothiazide ethanol, caffeine, theophylline, theobromine, amphotericin B, tolvaptan, conivaptan, dopamine, acetazolamide, dorzolamide. Examples of calcium channel blockers include amlodipine, aranidipine, azelnidipine, barnidipine, benidipine, cilnidipine, clevidipine, efonidipine, felodipine), isradipine, lacidipine, lercanidipine, manidipine, nicardipine, nifedipine, nilvadipine, nimodipine, nisoldipine, nitrendipine, pranidipine, fendiline, gallopamil, verapamil), and diltiazem. Examples of ACE inhibitors include captopril, zofenopril, enalapril, ramipril, quinapril, perindopril, lisinopril, benazepril, imidapril, trandolapril, cilazapril, and fosinopril. Examples of angiotensin II receptor antagonists include valsartan, telmisartan, losartan, irbesartan, irbesartan, azilsartan, and olmesartan. Examples of beta blockers include propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol. Butaxamine and nebivolol.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, N.Y., pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to the disclosed B7-H5 antibodies). In particular, such antibodies include immunoglobulin molecules of any type (e.g., IgG, IgE, IgM, IgD, IgA and IgY), class (e.g., IgG1, IgG2, IgG3, IgG4, IgA1 and IgA2) or subclass.

As used herein, the term “antigen binding fragment” of an antibody refers to one or more portions of an antibody that contain the antibody's Complementarity Determining Regions (“CDRs”) and optionally the framework residues that comprise the antibody's “variable region” antigen recognition site, and exhibit an ability to immunospecifically bind an antigen. Such fragments include Fab′, F(ab′)2, Fv, single chain (ScFv), and mutants thereof, naturally occurring variants, and fusion proteins comprising the antibody's “variable region” antigen recognition site and a heterologous protein (e.g., a toxin, an antigen recognition site for a different antigen, an enzyme, a receptor or receptor ligand, etc.).

Human, non-naturally occurring chimeric or humanized derivatives of UT-A urea transporter protein antibodies are particularly preferred for in vivo use in humans, however, murine antibodies or antibodies of other species may be advantageously employed for many uses (for example, in vitro or in situ detection assays, acute in vivo use, etc.). A humanized antibody may comprise amino acid residue substitutions, deletions or additions in one or more non-human CDRs. The humanized antibody derivative may have substantially the same binding, stronger binding or weaker binding when compared to a non-derivative humanized antibody. In specific embodiments, one, two, three, four, or five amino acid residues of the CDR have been substituted, deleted or added (i.e., mutated). Completely human antibodies are particularly desirable for therapeutic treatment of human subjects.

Human antibodies can be made by a variety of methods known in the art including phage display methods described above using antibody libraries derived from human immunoglobulin sequences (see U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741). Human antibodies can be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., all or a portion of a varicella-zoster virus polypeptide.

Monoclonal antibodies directed against the antigen can be obtained from the immunized, transgenic mice using conventional hybridoma technology (see, e.g., U.S. Pat. No. 5,916,771). The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93, which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

A “chimeric antibody” is a molecule in which different portions of the antibody are derived from different immunoglobulin molecules such that the entire molecule is not naturally occurring. Examples of chimeric antibodies include those having a variable region derived from a non-human antibody and a human immunoglobulin constant region. The term is also intended to include antibodies having a variable region derived from one human antibody grafted to an immunoglobulin constant region of a predetermined sequences or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. See Jefferis et al. mAb, 1 (2009), pp. 332-338.

### Pharmaceutical Compositions

In certain embodiments, this disclosure relates to pharmaceutical composition comprising a urea transporter inhibitor and a pharmaceutically acceptable excipient. In certain embodiments, the urea transporter inhibitor is N,N′-dimethylthiourea (DMTU), prodrug, derivative, or salt thereof. In certain embodiments, the urea transporter inhibitor is small molecule, protein, antibody or fragment thereof that specifically binds urea transport protein A and/or urea transport protein B. In certain embodiments, the pharmaceutical composition further comprises a statin, antihypertensive agent, diuretic, or combination thereof. In certain embodiments, the antihypertensive drug is an angiotensin receptor II blocker, angiotensin-converting enzyme inhibitor, calcium channel blocker, beta blocker, or combinations thereof. In certain embodiments, the diuretic is spironolactone or furosemide.

Pharmaceutical compositions typically comprise an effective amount of a compound or compounds disclosed herein and a suitable pharmaceutical acceptable carrier. The preparations can be prepared in a manner known per se, which usually involves mixing the compounds according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

The compound or compounds disclosed herein can be administered to a subject either alone or as a part of a pharmaceutical composition containing a pharmaceutically acceptable excipient. In certain embodiments, the pharmaceutical composition is in the form of a tablet, pill, capsule, gel, gel capsule or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, pharmaceutical composition is in solid form surrounded by an enteric coating. In certain embodiments, the enteric coating comprises methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, or combinations thereof.

The pharmaceutical compositions of the present disclosure can be administered to subjects either topically to the skin, orally, rectally, parenterally (intravenously, intramuscularly, or subcutaneously), intracisternally, intravaginally, intraperitoneally, intravesically, locally (powders, ointments, or drops), or as a buccal or nasal spray. Pharmaceutically acceptable salts, solvates, and hydrates of the compounds listed are also useful in the method of the disclosure and in pharmaceutical compositions of the disclosure.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain adjuvants such as preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar and as high molecular weight polyethylene glycols, and the like.

Solid dosage forms such as tablets, capsules, pills, and granules can be prepared with coatings and shells, such as enteric coatings and others well known in the art. They may contain opacifying agents and can also be of such composition that they release the active compound or compounds in a certain part of the intestinal tract in a delayed manner. Controlled slow release formulations are also preferred, including osmotic pumps and layered delivery systems.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions may contain suspending agents, as for example, ethoxylated iso-stearyl alcohols, polyoxyethylene sorbitol and sorbitan esters, microcrystalline cellulose, aluminum hydroxide, bentonite agar-agar and tragacanth, or mixtures of these substances, and the like.

Dosage forms for topical administration of a composition comprising (a compound or compounds disclosed herein include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this disclosure.

Pharmaceutical compositions typically comprise an effective amount of a compound or compounds disclosed herein and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet, 2006, 21(3):173-85, entitled “Human carboxylesterase isozymes: catalytic properties and rational drug design.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

### EXAMPLES

### Animal Experimental Protocol

Experiment of C57BL/6 wild type mice. Mice underwent 5/6 nephrectomy surgery. Mice were fed 14% protein diet (0 weeks to 2 weeks), 23% protein diet (2 weeks to 6 weeks), and 40% protein diet (6 weeks to 8 weeks). Mice drank 1% NaCl water one week to six weeks after second surgery, and dimethylthiourea (DMTU; UT-A inhibitor) was administered by osmotic mini pump.

### Cardiac Hypertrophy and Fibrosis Appeared in the 5/6 Nephrectomy Mouse Model

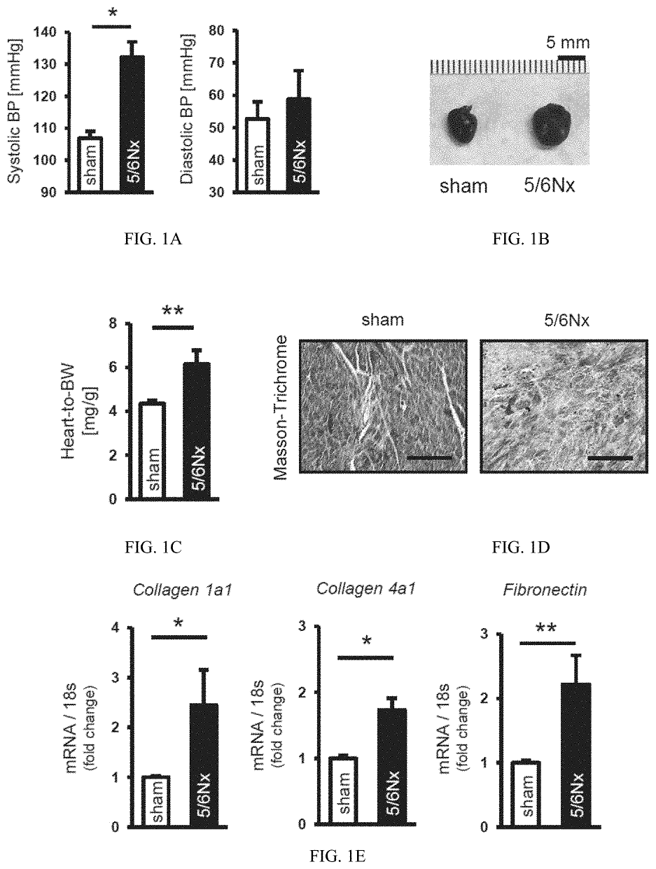

At eight weeks after 5/6Nx, the systolic BP (SBP) of the CKD mice significantly increased compared to sham mice (107 mmHg vs 132 mmHg, P<0.005). The diastolic BP (DBP) of the CKD mice (59 mmHg) trended toward an increase (53 mmHg), but did not reach a statistically significant difference (FIG. 1A). Heart size was increased in the 5/6Nx mice (FIG. 1B), and was accompanied by increased heart weight (4.35 mg/g vs 6.14 mg/g, P=0.028) (FIG. 1C). Cardiac fibrosis was identified in the heart of CKD mice by Masson's Trichrome staining (FIG. 1D). In addition, fibrosis related gene expression of collagen type 1 alpha (1A1), type 4A1 and fibronectin were increased in CKD hearts compared with the hearts of sham-operated mice (FIG. 1E). These data suggest that cardiac hypertrophy and fibrosis were induced in this animal model of CKD.

Cardiac Fibrosis in Uremic Heart was Associated with Upregulation of Ut-A and Vimentin Expression

It was reported that UT-A protein levels increase in uremic and hypertensive hearts from human and rat. To determine whether UT-A plays a role in uremic heart, UT-A protein abundance was first measured in the heart of mice. In normal C57BL/6J mouse heart, a 51-kDa band was detected by UT-A antibody; the band was completely ablated by pretreating the probing antibody with immunizing UT-A c-terminal peptide (FIG. 2A). The molecular weight of this band is consistent with UT-A2, which has an amino acid sequence identical to the c-terminal 397 amino acids of UT-A1. The larger UT-A1 protein, which runs at 117 and 97 kDa, was not detected in mouse heart. Second, UT-A-GFP was overexpressed in cultured H9c2 cells (rat heart myoblasts) by adenovirus-mediated gene transfer (FIG. 2B). Not only was UT-A protein increased, but vimentin, a pre-fibrosis protein, also increased in UT-A-GFP transduced cells (FIG. 2C). Similar results were found in the heart of CKD mice. The UT-A protein level increased 1.6-fold in the hearts of CKD mice and was associated with a 1.9-fold increased vimentin level (FIG. 2D). The increase in vimentin was confirmed by immunohistochemistry (FIG. 2E). These data suggest that UT-A protein might directly or indirectly raise vimentin in CKD, which promotes cardiac fibrosis.

### Inhibition of UT-A Altered the Hemodynamics in CKD Mice

Since an increase in UT-A is associated with an increase in fibrosis proteins in vitro and in vivo, experiments were performed to determine whether inhibition of UT-A could prevent fibrosis in the heart of CKD mice. Mice were randomly divided into four cohorts: sham+vehicle, sham+DMTU, 5/6Nx+vehicle, and 5/6Nx+DMTU. Mini-pumps were used for DMTU administration (100 mg/kgBW/day). The phenotypic characteristics of mice were determined 8 weeks after completing the 5/6Nx (Table 1).

TABLE 1
Phenotypic characteristics of WT and 5/6Nx mice

| Parameter | sham + vehicle | sham + DMTU | 5/6Dx + vehicle | 5/6Nx + DMTU |
|---|---|---|---|---|
| Initial BW (g) | 25.8 ± 0.4 | 25.0 ± 0.6 | 26.5 ± 0.6 | 26.0 ± 0.5 |
| Final BW (g) | 29.8 ± 0.4 | 25.9 ± 1.1* | 27.6 ± 0.7* | 26.4 ± 0.5* |
| BUN (mg/dl) | 33.6 ± 1.3 | 31.3 ± 2.8 | 69.9 ± 1.3*# | 63.7 ± 4.6*# |
| Urine urea (mmol/l) | 1510 ± 154 | 1555 ± 128 | 761 ± 48*# | 926 ± 95*# |
| 24 hr intake | 0.159 ± 0.028 | 0.129 ± 0.006 | 0.324 ± 0.012*# | 0.265 ± 0.028*# |
| volume/BW (ml/g) | | | | |
| 24 hr urine | 0.023 ± 0.004 | 0.058 ± 0.010* | 0.148 ± 0.008*# | 0.170 ± 0.013*# |
| volume/BW (ml/g) | | | | |
| Volume in-out | 0.145 ± 0.010 | 0.081 ± 0.010* | 0.185 ± 0.013# | 0.114 ± 0.014§ |
| balance/BW (ml/g) | | | | |
| 24 hr urine osmolality | 3595 ± 559 | 2378 ± 133* | 908 ± 40*# | 1206 ± 116*# |
| (mOsmol/kg H₂O) | | | | |
| Plasma osmolality | 322 ± 2 | 322 ± 1 | 330 ± 2 | 329 ± 3 |
| (mOsmol/kg H₂O) | | | | |

Blood urea nitrogen (BUN) levels in the 5/6Nx mice (both vehicle and DMTU treatment) were significantly higher than in sham mice verifying a functional decline in kidney function. Although the inhibition of UT-A blocks reabsorption of urea in the inner medulla collecting duct, there was no significant difference in BUN values with DMTU treatment vs. vehicle treatment in either sham or CKD mice. Inhibition of UT-A by DMTU increased urine volume and decreased urine osmolality in sham mice, but this was absent in CKD mice.

### Inhibition of UT-A by Dimethylthiourea (DMTU) Ameliorated CKD-Induced Hypertension and Cardiac Hypertrophy

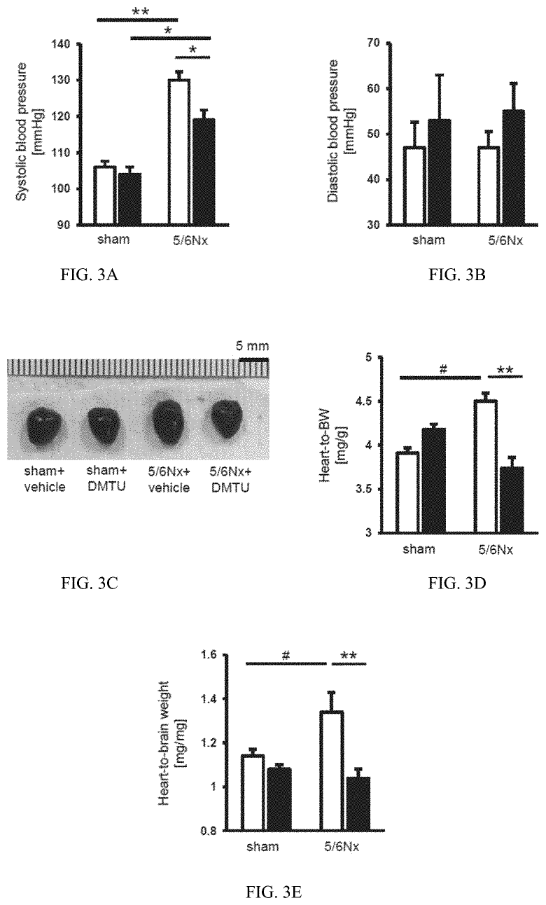

Inhibition of UT-A suppressed CKD-induced increases in SBP in 5/6Nx mice (5/6Nx+DMTU: 119±3 mmHg vs 5/6Nx+vehicle: 130±2 mmHg, P<0.05) (FIG. 3A). However, DMTU treatment did not affect DBP in either sham or 5/6Nx mice (FIG. 3B). DMTU suppressed the CKD-induced increase in heart size (FIG. 3C). The ratio of heart weight/body weight (BW) in 5/6Nx+vehicle (4.50±0.09 mg/g) was significantly increased compared with sham+vehicle mice (3.91±0.06 mg/g, P<0.005). DMTU suppressed the CKD-induced increase in heart weight (5/6Nx+DMTU: 3.74±0.12 mg/g vs 5/6Nx+vehicle: 4.50±0.09 mg/g, P<0.001) (FIG. 3D). Since the BW could be altered by either edema (in CKD) or dehydration (by DMTU), dry brain weight was also used to normalize heart weight. The ratio of heart weight to dry brain weight was significantly increased in 5/6Nx+vehicle (1.34±0.09 mg/mg) compared with sham+vehicle (1.14±0.03 mg/mg, P<0.05); and DMTU suppressed this increase (5/6Nx+DMTU: 1.04±0.04 mg/mg vs 5/6Nx+vehicle 1.34±0.09 mg/mg, P<0.01) (FIG. 3E). These results suggest that inhibition of UT-A with DMTU diminished CKD-induced hypertension and cardiac hypertrophy.

### Inhibition of UT-A Attenuated CKD-Induced Cardiac Fibrosis and Improved Heart Functions

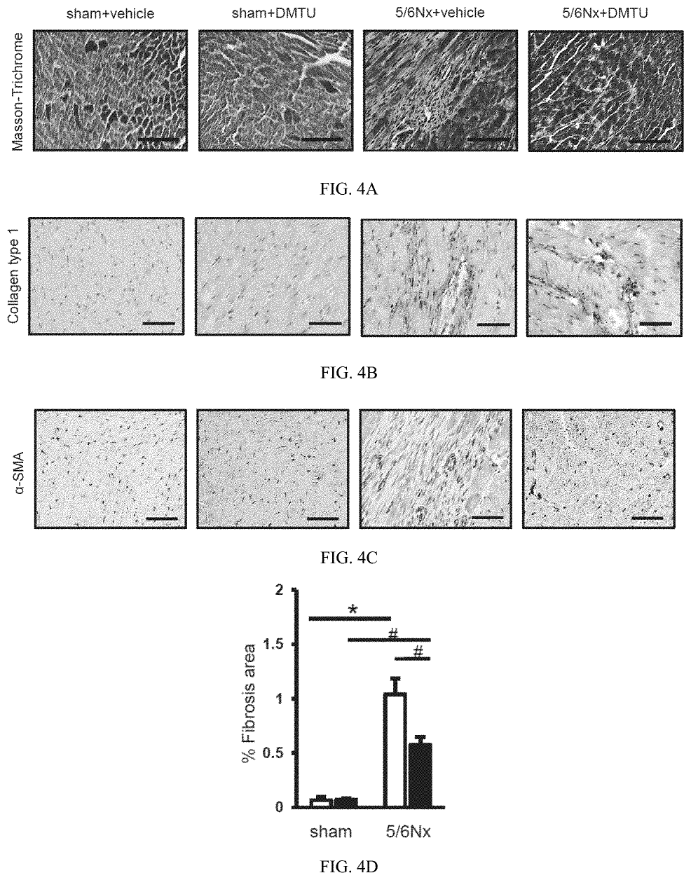

DMTU reduced the fibrosis in the hearts of CKD mice examined by Masson's staining. The percentage of fibrosis area in heart was sharply increased in 5/6Nx+vehicle (1.04±0.15%) vs sham+vehicle (0.07±0.03%), but DMTU suppressed the increase in fibrosis area in the CKD mouse (0.57±0.07%) (FIGS. 4A and 4D). By immunohistochemistry staining, collagen type I and α-smooth muscle actin (SMA) proteins increased in 5/6Nx+vehicle, but they were less in 5/6Nx+DMTU (FIGS. 4B and 4C). By quantitative PCR, mRNA expression of fibrosis related genes (collagen 1A1, collagen 4A1, fibronectin and α-SMA) in heart samples from 5/6Nx+DMTU mice were less than 5/6Nx+vehicle mice (FIG. 4E).

To identify whether UT inhibition had an impact on heart function, a cardiac echocardiographic analysis was performed. Both intraventricular septum (IVS) and posterior wall thicknesses (PW) were greatest in 5/6Nx+vehicle than all other three groups (Table 2).

TABLE 2
Echocardiogram analysis of mice after 5/6 nephrectomy and DMTU treatment

| | sham + vehicle | sham + DMTU | 5/6Nx + vehicle | 5/6Nx + DMTU |
|---|---|---|---|---|
| BW (g) | 29.9 ± 0.4 | 25.8 ± 1.2 | 27.1 ± 0.8 | 25.8 ± 0.6 |
| IVSs (mm) | 1.05 ± 0.00 | 1.05 ± 0.00 | 1.17 ± 0.00* | 1.08 ± 0.00^{#} |
| IVSd (mm) | 0.47 ± 0.02 | 0.44 ± 0.00 | 0.51 ± 0.01 | 0.49 ± 0.01 |
| PWs (mm) | 0.50 ± 0.02 | 0.52 ± 0.03 | 0.53 ± 0.01 | 0.52 ± 0.01 |
| PWd (mm) | 0.54 ± 0.02 | 0.53 ± 0.00 | 0.59 ± 0.01 | 0.55 ± 0.01 |
| LVESD (mm) | 2.37 ± 0.13 | 2.45 ± 0.14 | 3.34 ± 0.19* | 2.77 ± 0.02^{#} |
| LVEDD (mm) | 3.69 ± 0.12 | 3.80 ± 0.18 | 3.43 ± 0.18 | 3.43 ± 0.06 |
| FS (%) | 32 ± 1.5 | 33 ± 0.5 | 27 ± 0.5* | 30 ± 0.5^{#} |
| LVESV (μl) | 22.4 ± 1.4 | 21.8 ± 1.1 | 28.4 ± 0.4* | 23.6 ± 0.3^{#} |
| LVEDV (μl) | 57.5 ± 1.0 | 57.3 ± 1.8 | 66.6 ± 0.6* | 62.6 ± 0.3^{#} |
| LVM (mg) | 89 ± 4 | 97 ± 7 | 113 ± 8* | 89 ± 3^{#} |
| LVMI (mg/g) | 2.97 ± 0.09 | 3.73 ± 0.15 | 4.18 ± 0.27* | 3.44 ± 0.13^{#} |

Left ventricular (LV) end-systolic dimension (LVESD), LV end-diastolic volume (LVEDV), LV end-systolic volume (LVESV) and left ventricular mass index (LVMI: the ratio of LVM to BW) were significantly increased in 5/6Nx+vehicle compared with sham+vehicle group, treatment with 5/6Nx+DMTU eliminated these increases (Table 2). In addition, the percentage of fractional shortening (FS) was decreased in CKD heart, which indicates an impaired efficiency of the heart in ejecting blood. DMTU restored FS to the normal range. In some of 5/6Nx+vehicle mice, a pericardial effusion occurred. However, none of the mice in the other three cohorts showed evidence of a pericardial effusion. These data suggest that the intervention of DMTU suppressed cardiac hypertrophy and improved heart function in CKD mice.

### Inhibition of UT-A Attenuated CKD-Induced Upregulation of the Renin-Angiotensin System (RAS):

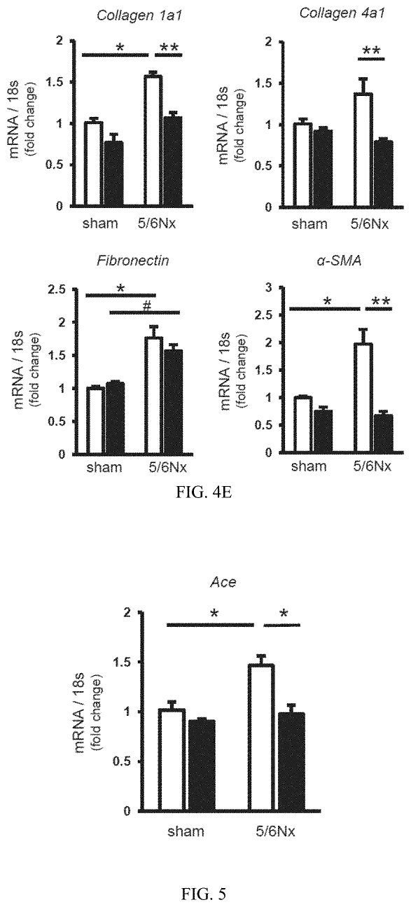

In uremic cardiomyopathy, increased cardiac fibrosis and hypertrophy are associated with upregulation of the renin-angiotensin system (RAS). To identify whether the ability of UT-A to reduce cardiac fibrosis is related to the RAS, the mRNA expression of angiotensin converting enzyme (ACE) was measured in the heart of 4 groups of mice. The mRNA level of Ace was sharply increased in 5/6Nx heart; DMTU treatment significantly abolished ACE expression in both sham and 5/6Nx group (FIG. 5). These data suggest that DMTU's ability to limit cardiac fibrosis may be related to reduce renin-angiotensin system.

1. A method of treating or preventing a cardiovascular disease or condition comprising administering an effective amount of a urea transporter inhibitor to a subject in need thereof.

2. The method of claim 1, wherein the subject is diagnosed with kidney disease.

3. The method of claim 2, wherein the subject is diagnosed with uremic cardiomyopathy.

4. The method of claim 1, wherein the urea transporter inhibitor is N,N′-dimethylthiourea (DMTU), prodrug, derivative, or salt thereof.

5. The method of claim 1, wherein the urea transporter inhibitor is small molecule, protein, antibody or fragment thereof that specifically binds urea transport protein A and/or urea transport protein B.

6. The method of claim 1 wherein the urea transporter inhibitor is administered in combination with statin, antihypertensive agent, diuretic, or combination thereof.

7. The method of claim 6 wherein the diuretic is spironolactone or furosemide.

8. The method of claim 6 wherein the antihypertensive agent is an angiotensin receptor II blocker, angiotensin-converting enzyme inhibitor, calcium channel blocker, beta blocker, or combinations thereof.

9. The method of claim 1, wherein the subject is over 50, 60, or 65 years of age.

10. A pharmaceutical composition comprising a urea transporter inhibitor and a pharmaceutically acceptable excipient.

11. The pharmaceutical composition of claim 10, wherein the urea transporter inhibitor is N,N′-Dimethylthiourea (DMTU), prodrug, derivative, or salt thereof.

12. The pharmaceutical composition of claim 10, wherein the urea transporter inhibitor is small molecule, protein, antibody or fragment thereof that specifically binds urea transport protein A and/or urea transport protein B.

13. The pharmaceutical composition of claim 10, wherein further comprising statin, antihypertensive agent, diuretic, or combination thereof.

14. The pharmaceutical composition of claim 13, wherein the antihypertensive drug is an angiotensin receptor II blocker, angiotensin-converting enzyme inhibitor, calcium channel blocker, beta blocker, or combinations thereof.

15. The pharmaceutical composition of claim 13, wherein the diuretic is spironolactone or furosemide.
